(19) United States
(12) Patent Application Publication
(10) Pub. No.: US 2007/0083240 A1
(43) Pub. Date: Apr. 12, 2007

(54) Methods and systems for applying stimulation and sensing one or more indicators of cardiac activity with an implantable stimulator

(76) Inventors: David K. L. Peterson, Saugus, CA (US); Kerry Bradley, Glendale, CA (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/636,146

(22) Filed: Dec. 7, 2006

Related U.S. Application Data
(63) Continuation-in-part of application No. 10/839,476, filed on May 5, 2004, now Pat. No. 7,162,304.
(60) Provisional application No. 60/469,084, filed on May 8, 2003.

(51) Int. Cl. A61N 1/18

(52) U.S. Cl. 607/2

(57) ABSTRACT

Methods of treating a medical disorder include implanting within a patient a stimulator with a number of electrodes electrically coupled thereto, applying a stimulation current via one or more of the electrodes to a stimulation site within the patient, and sensing one or more indicators of cardiac activity with one or more of the electrodes. Systems for treating a medical disorder include a stimulator configured to be implanted at least partially within a patient and to generate a stimulation current in accordance with one or more stimulation parameters adjusted to treat the medical disorder and a plurality of electrodes electrically coupled to the stimulator. One or more of the electrodes are configured to apply the stimulation current to one or more stimulation sites within the patient and one or more of the electrodes are configured to sense one or more indicators of cardiac activity.

### RELATED APPLICATIONS

The present application is a continuation-in-part application of U.S. application Ser. No. 10/839,476, filed May 5, 2004, which application claims the benefit of Provisional Application Ser. No. 60/469,084, filed May 8, 2003. Both applications are incorporated herein by reference in their entireties.

### BACKGROUND

Chronic pain is a major public health problem. It is estimated that chronic pain affects fifteen to thirty-three percent of the United States population or as many as seventy million people. Chronic pain disables more people than cancer or heart disease and costs Americans more than both diseases combined.

Neuropathic pain is one type of chronic pain that is caused by a malfunction somewhere in the nervous system. The site of the nervous system injury or malfunction can be either in the peripheral or central nervous system. Neuropathic pain is often triggered by a disease or an injury.

Patients with chronic pain currently have very few treatment alternatives. Chronic pain is often poorly controlled by medication. Surgery is often ineffective, as the pain may persist even after surgery. Chronic pain may also be controlled through the use of a transcutaneous electrical nerve stimulation (TENS) system which masks local pain sensations with a tingling sensation. However, TENS devices can produce significant discomfort and can only be used intermittently.

Spinal cord stimulation has also been used to treat chronic pain. Spinal cord stimulator (SCS) systems generate electrical pulses, also referred to as stimulation pulses, that are applied to one or more stimulation sites within a patient with chronic pain. The stimulation pulses are typically configured to mask the pain felt by a patient by producing a tingling sensation, which is also known as paresthesia.

Patients with chronic pain often have a reduced capacity to exercise as well as limited physical movement. Hence, patients being treated for chronic pain with, for example, spinal cord stimulation, are often encouraged to increase their physical activity throughout the treatment process. However, it is currently difficult to consistently and objectively monitor physical activity of a patient who is being treated for chronic pain.

### SUMMARY

Methods of treating a medical disorder include implanting within a patient a stimulator with a number of electrodes electrically coupled thereto, applying a stimulation current via one or more of the electrodes to a stimulation site within the patient, and sensing one or more indicators of cardiac activity with one or more of the electrodes.

Systems for treating a medical disorder include a stimulator configured to be implanted at least partially within a patient and to generate a stimulation current in accordance with one or more stimulation parameters adjusted to treat the medical disorder and a plurality of electrodes electrically coupled to the stimulator. One or more of the electrodes are configured to apply the stimulation current to one or more stimulation sites within the patient and one or more of the electrodes are configured to sense one or more indicators of cardiac activity.

### BRIEF DESCRIPTION OF THE DRAWINGS

The accompanying drawings illustrate various embodiments of the principles described herein and are a part of the specification. The illustrated embodiments are merely examples and do not limit the scope of the disclosure.

FIG. 1 illustrates an exemplary stimulator according to principles described herein.

FIGS. 2A-2B illustrate an exemplary spinal cord stimulator according to principles described herein.

FIG. 3 illustrates an exemplary microstimulator according to principles described herein.

FIG. 4 shows an example of a microstimulator with one or more leads coupled thereto according to principles described herein.

FIG. 5 shows a graphical representation of an exemplary EKG signal according to principles described herein.

FIG. 6 illustrates an exemplary stimulator configured to sense one or indicators of cardiac activity and apply electrical stimulation to one or more sites within a patient according to principles described herein.

FIGS. 7A-7B illustrate a number of exemplary electrode configurations that may be used with the stimulator of FIG. 6 according to principles described herein.

FIG. 8 illustrates an exemplary configuration wherein the stimulator is configured to perform impedance plethysmography of the thorax according to principles described herein.

Throughout the drawings, identical reference numbers designate similar, but not necessarily identical, elements.

### DETAILED DESCRIPTION

Methods and systems for treating one or more medical disorders are described herein. In some examples, a stimulator may be at least partially implanted within a patient and configured to generate a stimulation current in accordance with one or more stimulation parameters that are adjusted to treat a particular medical disorder. A plurality of electrodes, which may be disposed on one or more leads and/or on the outer surface of the stimulator, are electrically coupled to the stimulator. One or more of the electrodes are configured to apply the stimulation current to one or more stimulation sites within the patient and one or more of the electrodes are configured to sense one or more indicators of cardiac activity by, for example, sensing one or more EKG signals and/or performing impedance plethysmography. The sensed indicators of cardiac activity may then be used to assess the effectiveness of the stimulation and/or adjust the stimulation parameters to more effectively treat the medical disorder.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

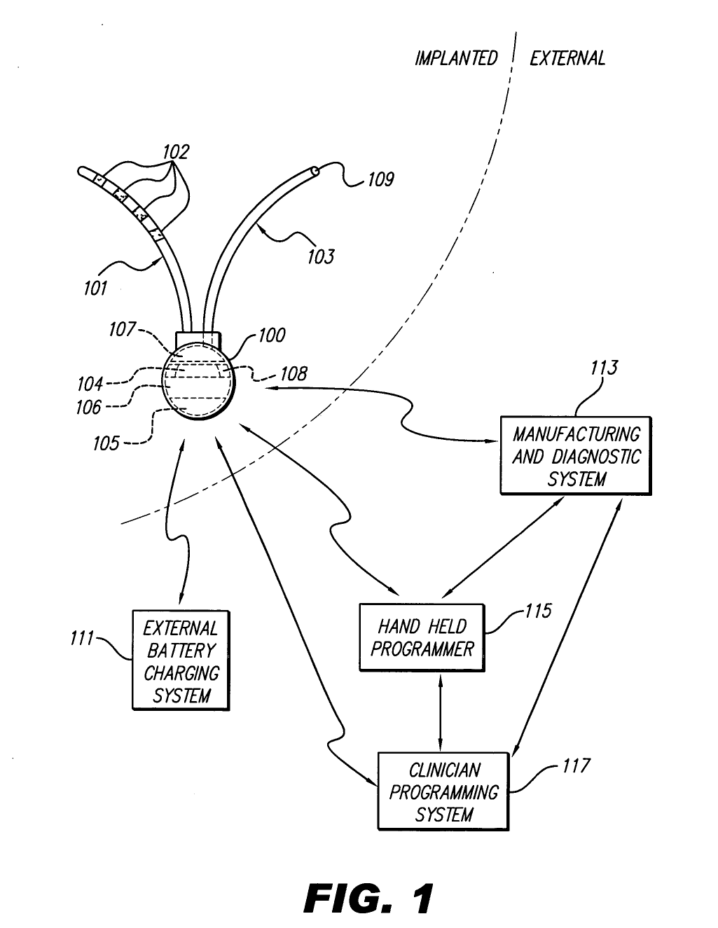

To facilitate an understanding of the methods and systems described herein, a more detailed description of a stimulator and its operation will now be given with reference to the figures. FIG. 1 illustrates an exemplary stimulator 100 that may be used to apply a stimulus to a stimulation site within a patient, e.g., an electrical stimulation of the stimulation site, an infusion of one or more drugs at the stimulation site, or both. The electrical stimulation function of the stimulator 100 will be described first, followed by an explanation of the possible drug delivery function of the stimulator 100. It will be understood, however, that the stimulator 100 may be configured to provide only electrical stimulation, only drug stimulation, both types of stimulation, or any other type of stimulation as best suits a particular patient.

The exemplary stimulator 100 shown in FIG. 1 is configured to provide electrical stimulation to one or more stimulation sites within a patient and may include a lead 101 coupled thereto. In some examples, the lead 101 includes a number of electrodes 102 through which electrical stimulation current may be applied to a stimulation site. It will be recognized that the lead 101 may include any number of electrodes 102 arranged in any configuration as best serves a particular application. A number of exemplary electrode configurations will be described herein in connection with a number of different figures. Hence, for illustrative purposes only, specific electrodes and electrode leads that are described in connection with the figures will each have unique reference numbers associated therewith.

As illustrated in FIG. 1, the stimulator 100 includes a number of components. It will be recognized that the stimulator 100 may include additional and/or alternative components as best serves a particular application. A power source 105 is configured to output voltage used to supply the various components within the stimulator 100 with power and/or to generate the power used for electrical stimulation. The power source 105 may include a primary battery, a rechargeable battery (e.g., a lithium-ion battery), a super capacitor, a nuclear battery, a mechanical resonator, an infrared collector (receiving, e.g., infrared energy through the skin), a thermally-powered energy source (where, e.g., memory-shaped alloys exposed to a minimal temperature difference generate power), a flexural powered energy source (where a flexible section subject to flexural forces is part of the stimulator), a bioenergy power source (where a chemical reaction provides an energy source), a fuel cell, a bioelectrical cell (where two or more electrodes use tissue-generated potentials and currents to capture energy and convert it to useable power), an osmotic pressure pump (where mechanical energy is generated due to fluid ingress), or the like.

In some examples, the power source 105 may be recharged using an external charging system. One type of rechargeable power supply that may be used is described in U.S. Pat. No. 6,596,439, which is incorporated herein by reference in its entirety. Other battery construction techniques that may be used to make the power source 105 include those shown, e.g., in U.S. Pat. Nos. 6,280,873; 6,458,171; 6,605,383; and 6,607,843, all of which are incorporated herein by reference in their respective entireties.

The stimulator 100 may also include a coil 108 configured to receive and/or emit a magnetic field (also referred to as a radio frequency (RF) field) that is used to communicate with, or receive power from, one or more external devices. Such communication and/or power transfer may include, but is not limited to, transcutaneously receiving data from the external device, transmitting data to the external device, and/or receiving power used to recharge the power source 105.

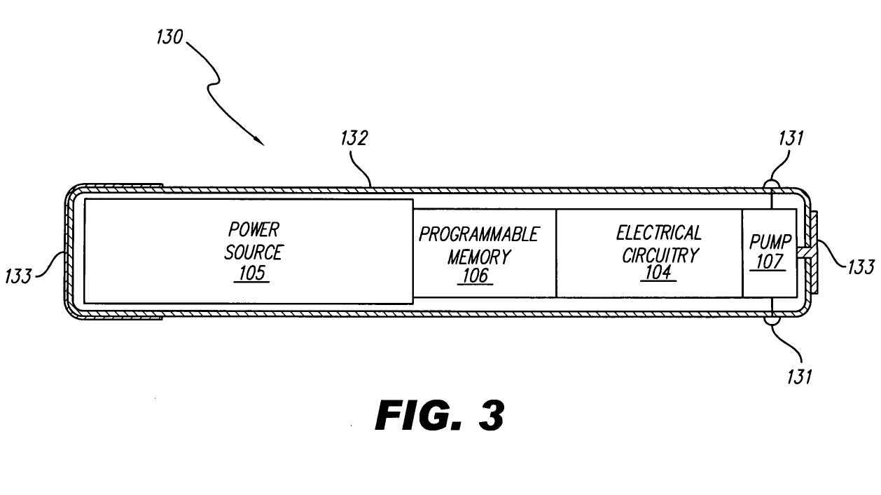

For example, an external battery charging system (EBCS) 111 may be provided to generate power that is used to recharge the power source 105 via any suitable communication link. Additional external devices including, but not limited to, a hand held programmer (HHP) 115, a clinician programming system (CPS) 117, and/or a manufacturing and diagnostic system (MDS) 113 may also be provided and configured to activate, deactivate, program, and/or test the stimulator 100 via one or more communication links. It will be recognized that the communication links shown in FIG. 3 may each include any type of link used to transmit data or energy, such as, but not limited to, an RF link, an infrared (IR) link, an optical link, a thermal link, or any other energy-coupling link.

Additionally, if multiple external devices are used in the treatment of a patient, there may be communication among those external devices, as well as with the implanted stimulator 100. It will be recognized that any suitable communication link may be used among the various devices illustrated.

The external devices shown in FIG. 1 are merely illustrative of the many different external devices that may be used in connection with the stimulator 100. Furthermore, it will be recognized that the functions performed by any two or more of the external devices shown in FIG. 1 may be performed by a single external device.

The stimulator 100 may also include electrical circuitry 104 configured to generate the electrical stimulation current that is delivered to a stimulation site via one or more of the electrodes 102. For example, the electrical circuitry 104 may include one or more processors, capacitors, integrated circuits, resistors, coils, and/or any other component configured to generate electrical stimulation current.

Additionally, the exemplary stimulator 100 shown in FIG. 1 may be configured to provide drug stimulation to a patient by applying one or more drugs at a stimulation site within the patient. To this end, a pump 107 may also be included within the stimulator 100. The pump 107 is configured to store and dispense one or more drugs, for example, through a catheter 103. The catheter 103 is coupled at a proximal end to the stimulator 100 and may have an infusion outlet 109 for infusing dosages of the one or more drugs at the stimulation site. In some embodiments, the stimulator 100 may include multiple catheters 103 and/or pumps 107 for storing and infusing dosages of the one or more drugs at the stimulation site.

The pump 107 described herein may include any of a variety of different drug delivery systems. For example, exemplary pumps 107 suitable for use as described herein include, but are not limited to, those disclosed in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,360,019; 4,487,603; 4,627,850; 4,692,147; 4,725,852; 4,865,845; 5,057,318; 5,059,423; 5,112,614; 5,137,727; 5,234,692; 5,234,693; 5,728,396; and 6,368,315. All of these listed patents are incorporated herein by reference in their respective entireties.

The stimulator 100 may also include a programmable memory unit 106 configured to store one or more stimulation parameters. The stimulation parameters may include, but are not limited to, electrical stimulation parameters, drug stimulation parameters, and other types of stimulation parameters. The programmable memory unit 106 allows a patient, clinician, or other user of the stimulator 100 to adjust the stimulation parameters such that the stimulation applied by the stimulator 100 is safe and efficacious for treatment of a particular patient. The programmable memory unit 106 may include any type of memory unit such as, but not limited to, random access memory (RAM), static RAM (SRAM), a hard drive, or the like.

The electrical stimulation parameters may control various parameters of the stimulation current applied to a stimulation site including, but not limited to, the frequency, pulse width, amplitude, waveform (e.g., square or sinusoidal), electrode configuration (i.e., anode-cathode assignment), burst pattern (e.g., burst on time and burst off time), duty cycle or burst repeat interval, ramp on time, and ramp off time of the stimulation current that is applied to the stimulation site. The drug stimulation parameters may control various parameters including, but not limited to, the amount of drugs infused at the stimulation site, the rate of drug infusion, and the frequency of drug infusion. For example, the drug stimulation parameters may cause the drug infusion rate to be intermittent, constant, or bolus. Other stimulation parameters that characterize other classes of stimuli are possible. For example, when tissue is stimulated using electromagnetic radiation, the stimulation parameters may characterize the intensity, wavelength, and timing of the electromagnetic radiation stimuli. When tissue is stimulated using mechanical stimuli, the stimulation parameters may characterize the pressure, displacement, frequency, and timing of the mechanical stimuli.

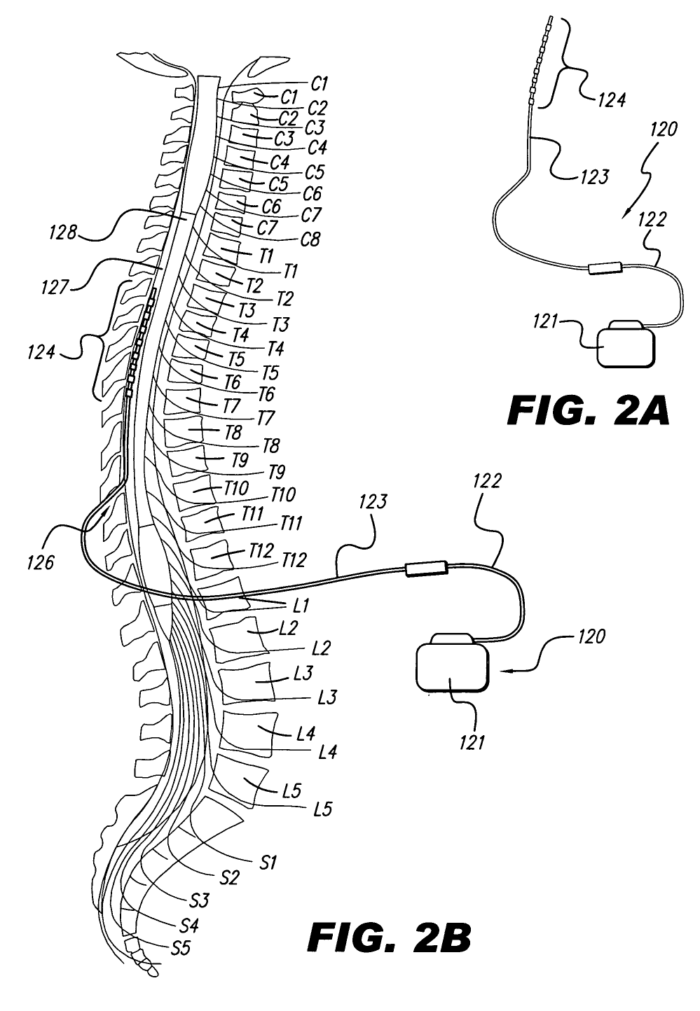

The stimulator 100 of FIG. 1 is illustrative of many types of stimulators that may be used in accordance with the systems and methods described herein. For example, FIGS. 2A-2B illustrate an exemplary spinal cord stimulator (SCS) 120 that may be used as the stimulator 100. The SCS 120 may be used to treat a number of different medical conditions such as, but not limited to, chronic pain, one or more movement disorders, and/or any other muscular and/or neural condition.

As shown in FIG. 2A, the SCS 120 may include an implantable pulse generator (IPG) 121, a lead extension 122, and a lead 123 having an array of electrodes 124 disposed thereon. The electrodes 124 may be arranged, as shown in FIG. 2A, in an in-line array near the distal end of the lead 123. Other electrode array configurations may additionally or alternatively be used. For example, the electrodes 124 may be arranged on a paddle lead. It will be recognized that the lead extension 122 is optional and that it may be used as desired depending on the physical distance between the IPG 121 and a desired stimulation site. The IPG 121 is configured to generate electrical stimulation pulses that are applied to a stimulation site via one or more of the electrodes 124. Exemplary spinal cord stimulators suitable for use as described herein include, but are not limited to, those disclosed in U.S. Pat. Nos. 5,501,703; 6,487,446; and 6,516,227, all of which are incorporated herein by reference in their respective entireties.

FIG. 2B shows that the array of electrodes 124 of the SCS 120 may be implanted in the epidural space 127 of a patient in close proximity to the spinal cord 128. Because of the lack of space near the lead exit point 126 where the electrode lead 123 exits the spinal column, the IPG 121 is generally implanted in the abdomen or above the buttocks. However, it will be recognized that the IPG 121 may be implanted at any suitable implantation site within a patient.

The stimulator 100 of FIG. 1 may alternatively include a microstimulator, such as a BION® microstimulator (Advanced Bionics® Corporation, Valencia, Calif.). Various details associated with the manufacture, operation, and use of implantable microstimulators are disclosed in U.S. Pat. Nos. 5,193,539; 5,193,540; 5,312,439; 6,185,452; 6,164,284; 6,208,894; and 6,051,017. All of these listed patents are incorporated herein by reference in their respective entireties.

FIG. 3 illustrates an exemplary microstimulator 130 that may be used as the stimulator 100 described herein. Other configurations of the microstimulator 130 are possible, as shown in the above-referenced patents and as described further below.

As shown in FIG. 3, the microstimulator 130 may include the power source 105, the programmable memory 106, the electrical circuitry 104, and the pump 107 described in connection with FIG. 1. These components are housed within a capsule 132. The capsule 132 may be a thin, elongated cylinder or any other shape as best serves a particular application. The shape of the capsule 132 may be determined by the structure of the desired stimulation site and the method of implantation. In some examples, the microstimulator 130 may include two or more leadless electrodes 133 disposed on the outer surface of the microstimulator 130.

The external surfaces of the microstimulator 130 may advantageously be composed of biocompatible materials. For example, the capsule 132 may be made of glass, ceramic, metal, or any other material that provides a hermetic package that will exclude water vapor but permit passage of electromagnetic fields used to transmit data and/or power. The electrodes 133 may be made of a noble or refractory metal or compound, such as platinum, iridium, tantalum, titanium, titanium nitride, niobium or alloys of any of these, in order to avoid corrosion or electrolysis which could damage the surrounding tissues and the device.

The microstimulator 130 may also include one or more infusion outlets 131 configured to dispense one or more drugs directly at a stimulation site. Alternatively, one or more catheters may be coupled to the infusion outlets 131 to deliver the drug therapy to a treatment site some distance from the body of the microstimulator 130.

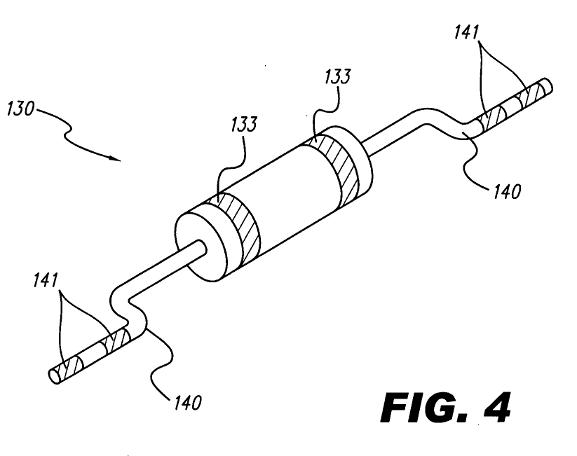

FIG. 4 shows an example of a microstimulator 130 with one or more leads 140 coupled thereto. As shown in FIG. 4, each of the leads 140 may include one or more electrodes 141 disposed thereon. As shown in FIG. 4, the microstimulator 130 may additionally or alternatively include one or more leadless electrodes 133 disposed on the outer surface thereof.

As mentioned, it may be desirable to monitor cardiac activity of a patient who is being treated for a medical disorder (e.g., chronic pain) with stimulation provided by an implantable stimulator 100. To this end, the stimulator 100 may additionally be configured to sense one or more indicators of cardiac activity. A physician or other care giver may then objectively monitor physical activity of a patient, gauge the effectiveness of the stimulation that is applied by the stimulator 100, and/or adjust the stimulation that is applied by the stimulator 100 to more effectively treat a particular medical condition (e.g., chronic pain).

It will be recognized that the stimulator 100 may be configured to sense one or more indicators of cardiac activity in connection with treating any type of medical condition as best serves a particular application. For example, the stimulator 100 may be configured to sense one or more indicators of cardiac activity in connection with treating chronic pain, one or more movement disorders, and/or any other muscular and/or neural condition.

As used herein, the term “one or more indicators of cardiac activity” will be used to refer to any indicator of cardiac activity that may be derived from an electrocardiogram (EKG), impedance plethysmography, and/or any other measurement that may be performed by the stimulator 100. Exemplary indicators of cardiac activity that may be derived from an EKG include, but are not limited to, heart rate, QT intervals, PR intervals, and heart abnormalities. Exemplary indicators of cardiac activity that may be derived from an impedance plethysmography include, but are not limited to, stroke volume of the heart, cardiac output, systemic vascular resistance, thoracic fluid content, pre-ejection period, left ventricular ejection time, systolic time ratio, left cardiac work index, and respiration rate. Each of these indicators of cardiac activity will be described in more detail below.

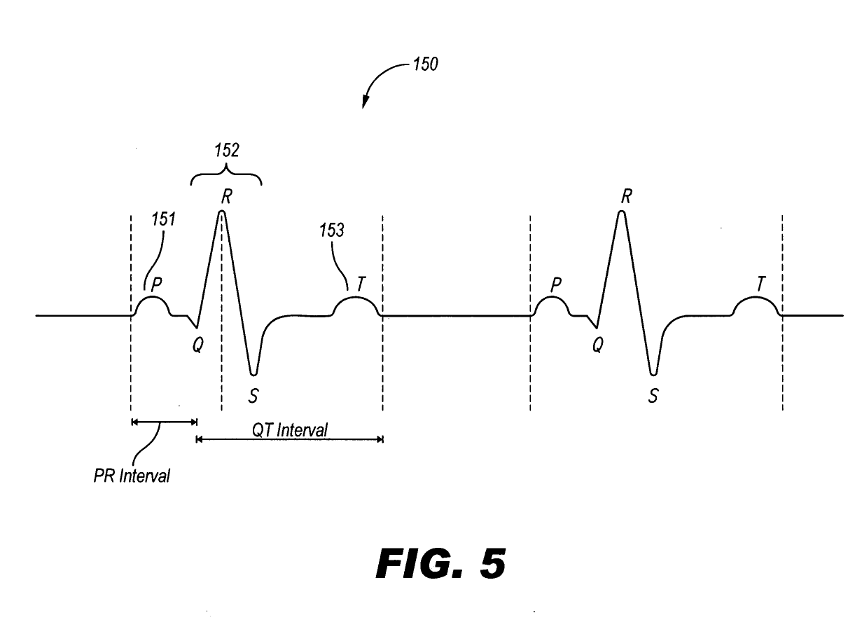

In some examples, the stimulator 100 may be configured to sense one or more indicators of cardiac activity by sensing one or more EKG signals. FIG. 5 shows a graphical representation of an exemplary EKG signal 150. The EKG signal 150 represents electrical activity within the heart. With each beat, electrical current travels through the heart and causes the heart to squeeze and pump blood therefrom.

As shown in FIG. 5, an EKG signal 150 corresponding to a normal heartbeat includes a P wave 151, a QRS complex 152, and a T wave 153. The P wave 151 corresponds to a portion of the current that causes atrial contraction. Both the left and right atria contract simultaneously. The relationship of the P wave 151 to the QRS complex 152, which will be described in more detail below, determines the presence of a heart block (a disease in the electrical system of the heart). An irregular or absent P wave 151 may be indicative of arrhythmia and/or one or more atrial problems.

The QRS complex 152 corresponds to a portion of the current that causes contraction of the left and right ventricles. Abnormalities in the QRS complex 152 may indicate bundle branch block, a ventricular origin of tachycardia, ventricular hypertrophy, or other ventricular abnormalities.

The T wave 153 represents the repolarization of the ventricles. Abnormalities in the T wave 153 may indicate electrolyte disturbance and can be a sign of cardiac disease.

As shown in FIG. 5, the QT interval is measured from the beginning of the QRS complex 152 to the end of the T wave 153. Abnormalities in the length of the QT interval may be indicators of heart disease and cardiac arrhythmia.

Also shown in FIG. 5 is the PR interval. The PR interval is measured from the beginning of the P wave 151 to the beginning of the QRS complex 152. A prolonged PR interval indicates a first degree heart block, while a shorting thereof may indicate an accessory bundle that depolarizes the ventricle undesirably early.

A number of additional or alternative indicators of cardiac activity may be derived from the EKG 150. For example, the heart rate may be derived by measuring the time in between successive R peaks 152. The reciprocal of this time is equal to the heart rate. The EKG 150 may also be used to detect cardiac arrhythmia, damage to the heart muscle (e.g., myocardial infarction), ischaemia of heart muscle (i.e., angina), electrolyte disturbances (e.g., potassium, calcium, and magnesium), and/or conduction abnormalities. The EKG 150 may also be used to provide information regarding the physical condition of the heart (e.g., left ventricular hypertrophy and/or mitral stenosis) and to screen for ischaemic heart disease during an exercise tolerance test.

The stimulator 100 described herein may additionally or alternatively be configured to sense one or more indicators of cardiac activity by performing impedance plethysmography, also referred to as impedance cardiography. Impedance plethysmography is a measurement technique that measures the change in blood volume for a specific body segment (e.g., the thorax). As the blood volume changes, the electrical impedance of the body segment also changes. This electrical impedance is measured by passing a small amount of alternating current (AC) through the body segment and detecting a change in voltage potential across the body segment as the heart beats. The ratio of voltage to current is equal to the impedance.

As mentioned, exemplary indicators of cardiac activity that may be derived from impedance plethysmography include, but are not limited to, stroke volume of the heart, cardiac output, systemic vascular resistance, thoracic fluid content, pre-ejection period, left ventricular ejection time, systolic time ratio, left cardiac work index, and respiration rate. Stroke volume refers to the amount of blood ejected from a ventricle with each beat of the heart and is often used to measure the overall activity of a patient. Cardiac output is the amount of blood the left ventricle ejects into systemic circulation in one minute. Systemic vascular resistance is the force that the ventricle must overcome to eject blood into the aorta. Thoracic fluid content is representative of total fluid volume in the chest, including both intra-vascular and extra-vascular fluid, and is calculated as the inverse of a baseline impedance measurement. Pre-ejection period is the period of isovolumetric ventricular contraction when the heart is pumping against a closed aortic valve. Left ventricular ejection time is the time between the opening and closing of the aortic valve. Systolic time ratio is the ratio of electrical to mechanical systole. Left cardiac work index is the amount of work the left ventricle performs each minute when ejecting blood. Respiration rate is a measurement of the amount of air going in and out of the lungs.

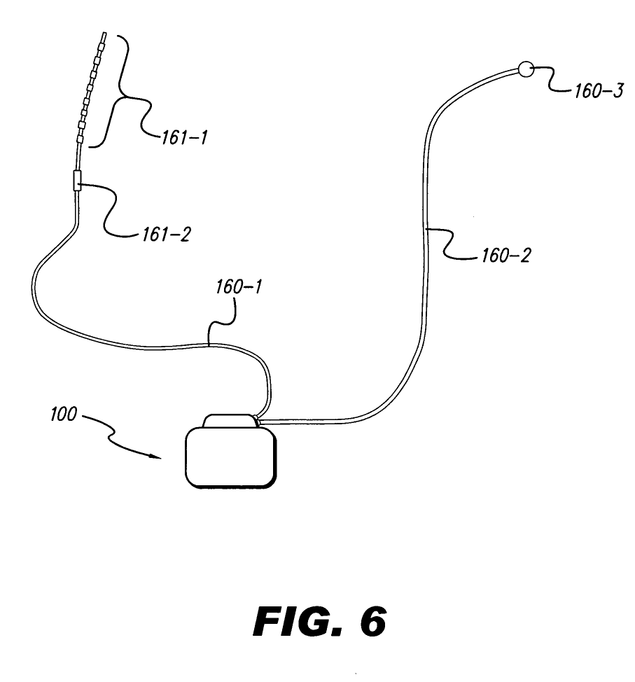

FIG. 6 illustrates an exemplary stimulator 100 configured to sense one or more indicators of cardiac activity and apply electrical stimulation to one or more sites within a patient. In some examples, as will be described in more detail below, the stimulator 100 of FIG. 6 may be configured to sense one or more indicators of cardiac activity by sensing one or more EKG signals. Additionally or alternatively, as will also be described in more detail below, the stimulator may be configured to sense one or more indicators of cardiac activity by performing impedance plethysmography.

As shown in FIG. 6, the stimulator 100 may have one or more leads (e.g., 160-1 and 160-2, collectively referred to herein as 160) coupled thereto. Each lead 160 may include one or more electrodes (e.g., 161-161-2, and 161-3, collectively referred to herein as 161) disposed thereon. As will be described in more detail below, one or more of the electrodes 161 shown in FIG. 6 may be configured to function as stimulating electrodes and one or more of the electrodes 161 may be configured to function as sensing electrodes. Additionally or alternatively, one or more of the electrodes 161 may be selectively configured to function as stimulating electrodes in some instances and as sensing electrodes in other instances. In this manner, the stimulator 100 may be configured to both apply electrical stimulation to one or more stimulation sites within a patient and sense one or more indicators of cardiac activity.

As shown in FIG. 6, a first lead 160-1 may include a plurality of electrodes 161 disposed thereon. In some examples, one or more of the electrodes (e.g., the electrodes labeled 161-1) are configured to function as stimulating electrodes. The first lead 160-1 may additionally include a dedicated electrode 160-2 configured to function as a sensing electrode. However, it will be recognized that each of the electrodes 161-1 and 161-2 may be selectively configured to function as a stimulating electrode in some instances and as a sensing electrode in other instances.

In some examples, a second lead 160-2 may additionally be coupled to the stimulator 100. As shown in FIG. 6, the second lead 160-2 may include a single electrode 161-3 configured to function as a sensing electrode. However, it will be recognized that the second lead 160-2 may alternatively include any number of electrodes 161 selectively configured to function as either stimulating electrodes or as sensing electrodes.

In some examples, one or more portions of the outer surface of the stimulator 100 may additionally or alternatively be configured to function as an indifferent electrode, as a stimulating electrode, or as a sensing electrode.

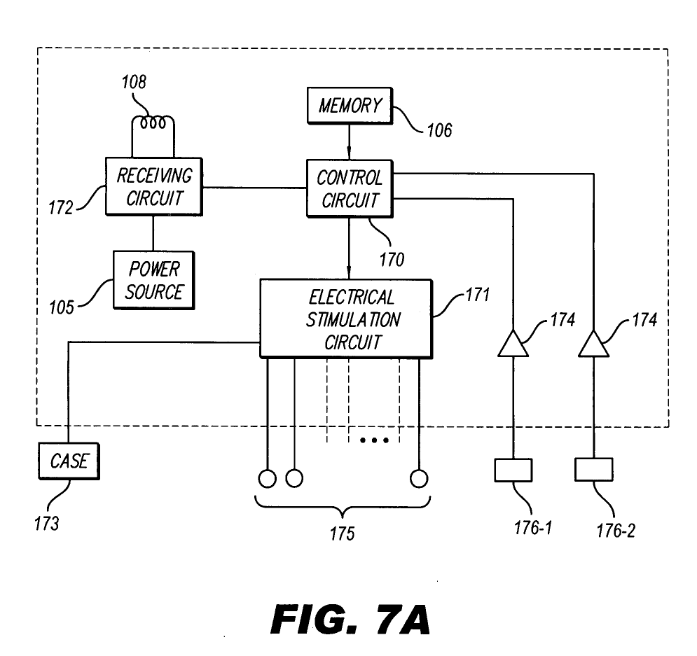

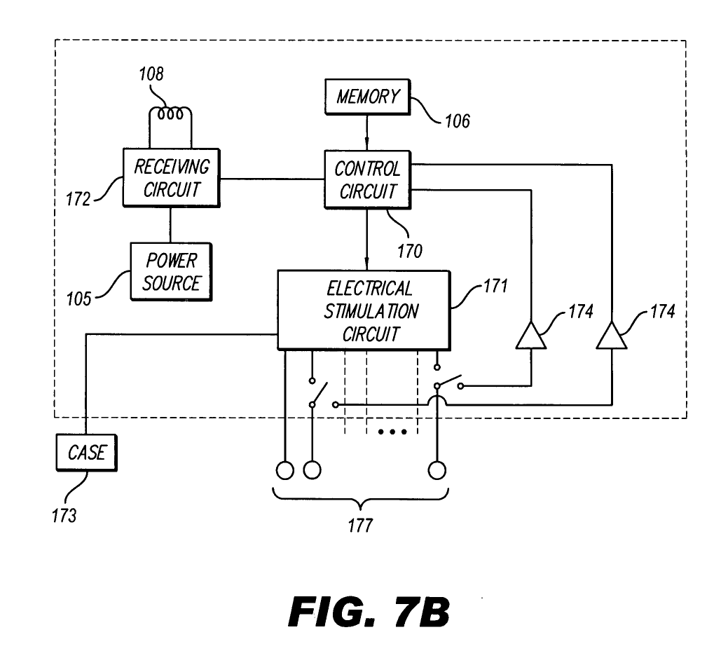

FIGS. 7A-7B illustrate a number of exemplary electrode configurations that may be used with the stimulator 100 of FIG. 6. It will be recognized that the components shown in FIGS. 7A-7B are merely illustrative and that additional or alternative components may be used.

As shown in FIG. 7A, the stimulator 100 may include a system control circuit 170 that is configured to control one or more of the other components within the stimulator 100. The system control circuit 170 may include any suitable processor or combination of hardware, software, and/or firmware as best serves a particular application.

In some examples, the system control circuit 170 is communicatively coupled to a memory unit 106 configured to store one or more stimulation parameters and to an electrical stimulation circuit 171 that is configured to generate electrical stimulation current in accordance with the stimulation parameters. The electrical stimulation circuit 171 may include any combination of components as best serves a particular application.

The stimulator 100 may also include a receiving circuit 171 coupled to a coil 108. The receiving circuit 171 is configured to receive power and/or data that is transmitted from one or more external devices and/or other implanted devices. The receiving circuit 171 may be communicatively coupled to an internal power source 105 configured to supply power to various components within the stimulator 100.

As shown in FIG. 7A, a number of electrodes 175 may be coupled to the electrical stimulation circuit 171. In some examples, the case 173, or outer surface of the stimulator 100, may also be configured to function as an electrode.

In some examples, each of the electrodes 175 is configured to function as stimulating electrodes and deliver electrical stimulation current to one or more stimulation sites within a patient. Each of the stimulating electrodes 175 is located on a single lead 101. Alternatively, a plurality of leads 101 each including one or more of the electrodes 175 may be coupled to the stimulator 100.

In some examples, one or more dedicated sensing electrodes (e.g., 176-1 and 176-2, collectively referred to herein as 176) may be coupled to one or more corresponding sense amplifiers 174 within the stimulator 100. Each sensing electrode 176 is configured to sense one or more indicators of cardiac activity and transmit the sensed information to the control circuit 170 for processing via the sense amplifiers 174. In some examples, the control circuit 170 may then transmit the sensed information to one or more external devices and/or additional implanted devices.

In some examples, one or more of the sensing electrodes 176 may be located on a dedicated sensing lead that is coupled to the stimulator 100. Additionally or alternatively, one or more of the sensing electrodes 176 may be located on a lead that also includes one or more of the stimulating electrodes 175.

FIG. 7B illustrates an alternative configuration of the stimulator 100 wherein one or more electrodes coupled thereto may be selectively configured to function as either stimulating electrodes or as sensing electrodes.

As shown in FIG. 7B, electrodes 177 are coupled to corresponding switches 178 that are located within the stimulator 100. Each switch 178 is configured to switch between a sense amplifier 174 and the electrical stimulation circuit 171. In this manner, one or more of the electrodes 177 may be configured to function as stimulating electrodes in some instances and as sensing electrodes in other instances.

In some examples, two or more of the electrodes described in connection with FIGS. 6-7B may be configured to sense one or more EKG signals. The two or more electrodes may be separated by any suitable distance and may sense the one or more EKG signals using any suitable method. In some examples, the sensed EKG signals are processed by the stimulator 100 and/or transmitted to an external device for processing and/or monitoring. One or more of the remaining electrodes may be configured to simultaneously or subsequently apply an electrical stimulation current to one or more stimulation sites within the body.

For example, electrodes 161-2 and 161-3 shown in FIG. 6 may be configured during a particular time period to function as sensing electrodes. The electrodes 161-2 and 161-3 may then sense one or more EKG signals during that time period and transmit the sensed signals to the stimulator 100. Electrical stimulation may simultaneously or subsequently be applied via one or more stimulating electrodes (e.g., electrodes 161-1).

Once one or more EKG signals have been obtained by the stimulator 100, the EKG signals may be analyzed by the stimulator 100 or by any other device configured to communicate with the stimulator 100 and used to assess the effectiveness of the stimulation and/or adjust the stimulation parameters such that the stimulation more effectively treats the patient.

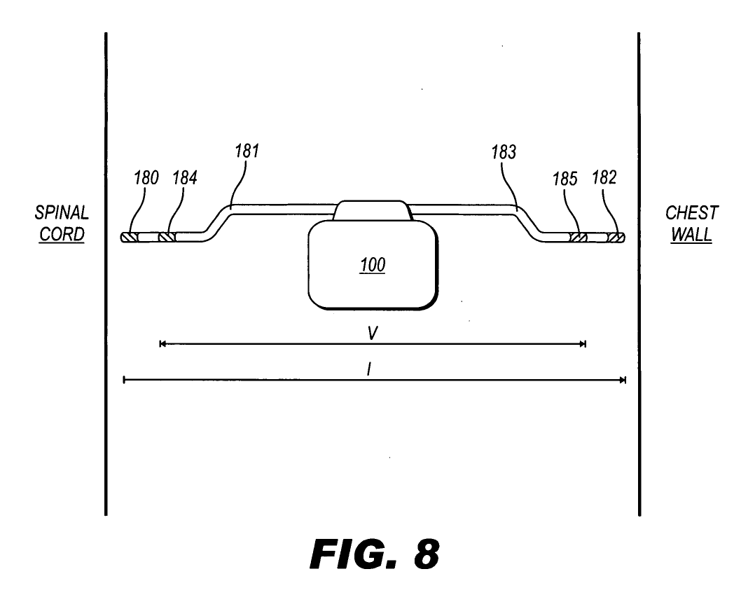

In some alternative examples, a number of the electrodes described in connection with FIGS. 6-7B may be configured to perform impedance plethysmography of the thorax in order to sense one or more indicators of cardiac activity. For example, FIG. 8 illustrates an exemplary configuration wherein the stimulator 100 is configured to perform impedance plethysmography of the thorax. Because the thorax is filled with blood, which is conductive, and air, which is resistive, changes in the amount of air or blood contained therein may be extracted by examining the impedance across at least a portion of the thorax.

Hence, as shown in FIG. 8, the stimulator 100 may be placed within the patient such that at least one electrode (e.g., electrode 180) is in communication with a stimulation site (e.g., the spinal cord). As used herein, the term “in communication with” refers to an electrode being adjacent to, in the general vicinity of, in close proximity to, directly next to, or directly on the stimulation site. The stimulation site of FIG. 8 is the spinal cord for illustrative purposes only. It will be recognized that the stimulation site may include additional or alternative locations within the patient as best serves a particular application.

As shown in FIG. 8, electrode 180 may be disposed on a first lead 181 that is coupled to the stimulator 100. Additionally or alternatively, the electrode 180 may be disposed on the surface of the stimulator 100.

In some examples, at least one additional electrode 182 configured to function as a stimulating electrode may be positioned such that it is in communication with the chest wall. Electrode 182 may be disposed on a second lead 183, as shown in FIG. 8. In some alternative examples, electrode 182 is disposed on the first lead 181 or on the outer surface of the stimulator 100.

Current may then be generated by the stimulator 100 and passed between the two electrodes 180 and 182, as shown in FIG. 8. The current is represented in FIG. 8 by the line labeled 1. It will be recognized that the direction of the current flow may vary as best serves a particular application. The current I may have a relatively low amplitude (e.g., less than or equal to 1 mA), a relatively low pulse width (e.g., less than or equal to 30 microseconds), and any suitable frequency (e.g., between about 20-100 kHz). However, it will be recognized that the current may have any combination of amplitude, pulse width, and frequency as best serves a particular application.

While the current is being applied between electrodes 180 and 182, two separate electrodes 184 and 185 may be used to sense a voltage potential V therebetween as the heart beats. As shown in FIG. 8, electrode 184 may be disposed on the first lead 181 and electrode 185 may be disposed on the second lead 183. However, it will be recognized that electrodes 184 and 185 may alternatively be disposed on one or more additional or alternative leads. It will also be recognized that electrodes 184 and 185 may be placed at any suitable location within the thorax. Moreover, it will also be recognized that the outer surface of the stimulator 100 may additionally or alternatively be used as one of the electrodes that senses the voltage potential V within the thorax.

The impedance (Z) of the thorax may be obtained by taking the ratio of the measured voltage potential between electrodes 184 and 185 to the current applied between electrodes 180 and 182. In other words, Z=V/I. As blood and air go in and out of the thorax, the impedance of the thorax changes. By sensing these changes in impedance, one or more of the indicators of cardiac activity described above may be derived.

Once the impedance measurements have been obtained by the stimulator 100, the impedance measurements may be analyzed by the stimulator 100 or by any other device configured to communicate with the stimulator 100 and used to assess the effectiveness of the stimulation and/or adjust the stimulation parameters such that the stimulation more effectively treats the patient.

In some examples, the stimulator 100 of FIG. 6 may be configured to operate independently. Alternatively, the stimulator 100 may be configured sense one or more indicators of cardiac activity by operating in a coordinated manner with one or more additional stimulators, other implanted devices, or other devices external to the patient's body.

For example, one or more sensor devices may additionally be implanted within the body and configured to operate in connection with the stimulator 100 described herein. The one or more sensor devices may include, but are not limited to, one or more pressure sensors. For example, one or more pressure sensors may be placed such that they are in communication with the heart, one or more veins or arteries, or the lungs. The pressure sensors may be configured to sense ventricular pressure and/or any other type of pressure associated with the cardiac system.

The sensed pressure information may then be communicated to the stimulator 100 and/or to any other device configured to operate in connection with the stimulator 100. The pressure information may then be analyzed and used to assess the effectiveness of the stimulation and/or adjust the stimulation parameters such that the stimulation more effectively treats the patient.

The preceding description has been presented only to illustrate and describe embodiments of the invention. It is not intended to be exhaustive or to limit the invention to any precise form disclosed. Many modifications and variations are possible in light of the above teaching.

What is claimed is:

1. A method comprising:
implanting a stimulator within a patient, said stimulator having a plurality of electrodes electrically coupled thereto;
applying a stimulation current via one or more of said electrodes to a stimulation site within said patient; and
sensing one or more indicators of cardiac activity with one or more of said electrodes.

2. The method of claim 1, wherein said one or more electrodes that sense said one or more indicators of cardiac activity are configured to sense at least one electrocardiogram (EKG) signal.

3. The method of claim 1, wherein said one or more electrodes that sense said one or more indicators of cardiac activity are configured to perform impedance plethysmography of the thorax.

4. The method of claim 3, wherein said one or more electrodes that are configured to perform impedance plethysmography comprise a first electrode, a second electrode, a third electrode, and a fourth electrode, and wherein said method further comprises:
passing an electrical current between said first and second electrodes; and
sensing a change in voltage potential within said thorax with said third and fourth electrodes while said electrical current is being passed between said first and second electrodes.

5. The method of claim 1, wherein said one or more indicators of cardiac activity comprise at least one or more of a heart rate, QT interval, PR interval, heart abnormality, stroke volume, cardiac output, systemic vascular resistance, thoracic fluid content, pre-ejection period, left ventricular ejection time, systolic time ratio, left cardiac work index, and respiration rate.

6. The method of claim 1, wherein said plurality of electrodes comprises at least one electrode configured to be selectively programmed to function as a stimulating electrode or as a sensing electrode.

7. The method of claim 1, further comprising assessing a physical activity level of said patient based on said sensed indicators of cardiac activity.

8. The method of claim 1, further comprising adjusting said stimulation current in accordance with said sensed indicators of cardiac activity.

9. The method of claim 1, further comprising infusing one or more drugs at said stimulation site with said stimulator.

10. A method of treating a medical disorder, said method comprising:
implanting a stimulator within a patient, said stimulator having a plurality of electrodes electrically coupled thereto;
applying a stimulation current via one or more of said electrodes to a stimulation site within said patient in accordance with one or more stimulation parameters configured to treat said medical disorder;
sensing one or more indicators of cardiac activity with one or more of said electrodes; and
adjusting one or more of said stimulation parameters in accordance with said sensed indicators of cardiac activity.

11. The method of claim 10, wherein said one or more electrodes that sense said one or more indicators of cardiac activity are configured to sense at least one electrocardiogram (EKG) signal.

12. The method of claim 10, wherein said one or more electrodes that sense said one or more indicators of cardiac activity are configured to perform impedance plethysmography of the thorax.

13. The method of claim 10, wherein said one or more indicators of cardiac activity comprise at least one or more of a heart rate, QT interval, PR interval, heart abnormality, stroke volume, cardiac output, systemic vascular resistance, thoracic fluid content, pre-ejection period, left ventricular ejection time, systolic time ratio, left cardiac work index, and respiration rate.

14. The method of claim 10, wherein said plurality of electrodes comprises at least one electrode configured to be selectively programmed to function as a stimulating electrode or as a sensing electrode.

15. The method of claim 10, wherein said medical disorder comprises at least one or more of chronic pain and a movement disorder.

16. A system for treating a medical disorder, said system comprising:
a stimulator configured to be implanted at least partially within a patient and to generate a stimulation current in accordance with one or more stimulation parameters adjusted to treat said medical disorder; and
a plurality of electrodes electrically coupled to said stimulator;
wherein one or more of said electrodes are configured to apply said stimulation current to one or more stimulation sites within said patient; and
wherein one or more of said electrodes are configured to sense one or more indicators of cardiac activity.

17. The system of claim 16, wherein said one or more electrodes that sense said one or more indicators of cardiac activity are configured to sense at least one electrocardiogram (EKG) signal.

18. The system of claim 16, wherein said one or more electrodes that sense said one or more indicators of cardiac activity are configured to perform impedance plethysmography of the thorax.

19. The system of claim 18, wherein said one or more electrodes that are configured to perform impedance plethysmography comprise:
a first electrode and a second electrode configured to pass an electrical current therebetween; and
a third electrode and a fourth electrode configured to sense a change in voltage potential within said thorax while said electrical current is being passed between said first and second electrodes.

20. The system of claim 16, wherein said one or more indicators of cardiac activity comprise at least one or more of a heart rate, QT interval, PR interval, heart abnormality, stroke volume, cardiac output, systemic vascular resistance, thoracic fluid content, pre-ejection period, left ventricular ejection time, systolic time ratio, left cardiac work index, and respiration rate.
